(19) United States
(12) Patent Application Publication
(10) Pub. No.: US 2021/0042481 A1
(43) Pub. Date: Feb. 11, 2021

(54) Microchip Denture Implant And Reader System And Method

(71) Applicant: Vahid Danaei-Moghaddam, Woodbridge (CA)

(72) Inventor: Vahid Danaei-Moghaddam, Woodbridge (CA)

(21) Appl. No.: 17/083,429

(22) Filed: Oct. 29, 2020

Related U.S. Application Data
(63) Continuation-in-part of application No. 16/387,511, filed on Apr. 17, 2019, now Pat. No. 10,872,212.
Application No. 16/387,511 is a continuation of application No. 15/807,863, filed on Nov. 9, 2017, now abandoned.

(51) Int. Cl. G06K 7/10; G06K 19/07; A61C 13/00

(52) U.S. Cl. CPC G06K 7/10366; G06K 19/0723; H04B 1/40; A61C 2204/005; A61C 13/00

(57) ABSTRACT

A microchip denture implant kit, system, and method. The kit, system, and method include a microchip having a transmitter for transmitting a wireless signal containing information relating to a user or a dental prosthetic, and an external reader scanner in communication with the microchip. The microchip mounts in a dental prosthetic item for oral use. The external reader when activated can scan and display or otherwise provide access to the information relating to the user. The microchip and dental prosthetic item, when installed in the mouth of the user, are configured to provide access to the information relating to the user at a given time.

### CROSS REFERENCE TO RELATED APPLICATION

The present application is a continuation application of pending application Ser. No. 16/387,511 filed on Apr. 17, 2019, which is a continuation-in-part of application Ser. No. 15/807,863 filed Nov. 9, 2017, which are both incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is explicitly or implicitly referenced is prior art.

### 1. FIELD OF THE INVENTION

The present invention generally relates to the field of dentistry and more specifically relates to dental accessories and tracking devices.

### 2. DESCRIPTION OF RELATED ART

Numerous electronic devices have been introduced to track and locate mobile assets, such as trucks, rail cars, and shipping containers. Hundreds of thousands of these assets have been equipped with tracking and locating transducers. The most sophisticated systems permit location of an asset to within a few feet. Tracking devices, such as radio frequency identification tags (RFID), are increasingly used to mark items to provide a convenient means of determining the location and tracking the movement of the article on which the tag is mounted. Such RFID tags or microchips are also increasingly used to provide identification and location information on people and animals. However, a reliable method of applying and using a microchip has not been created for many items of value or for tracking important items/people of concern. A suitable, affordable, noninvasive solution is desired.

U.S. Pub. No. 2009/0237236 to Sarni Maassarani relates to a tooth locate GPS person tracking and location method and apparatus. The described tooth located GPS person tracking and location method and apparatus includes an apparatus and method for remotely monitoring the location of a person through a microchip mounted in a tooth of a person which communicates by a communication link to a Global Positioning System to obtain geographic location information relating to the physical location of a person, and by another communication link to a remote tracking device to obtain the geographic information to determine and provide an indication of the physical location of a person. The microchip is disposed in a cavity formed in a tooth of a person which is filled with dental filling material to fix the microchip in the tooth.

### BRIEF SUMMARY OF THE INVENTION

Given the foregoing disadvantages inherent in the known dentistry art, the present disclosure provides a novel microchip denture implant and reader system and method. The general purpose of the present disclosure, which will be described subsequently in greater detail, is to provide a personalized, microchip denture implant which will be processed inside part of any type of prosthetics wearable inside a user's mouth and may be accessed by an external reader device.

A microchip denture implant and reader system are disclosed herein. The microchip denture implant and reader system include a microchip including a transceiver 1150 for transmitting a wireless signal and an external reader scanner 130 in communication with the microchip. The wireless signal can contain information relating to a user of dentures or other dental prosthetics. The microchip is adapted to be mounted in a dental prosthetic item to be applied to the mouth of the user. The external reader when activated is configured to scan and provide access to the information relating to the user by displaying it. The microchip and dental prosthetic item, when installed in the mouth of the user, are configured to provide access to the information relating to the user at a given time.

The dental prosthetic item may include a partial denture, orthodontic, dental retainers, or orthopedic appliance, a complete fixed over dental implant supported or complete removable or over implant supported denture. The device may be used to locate, identify, or retrieve personal information on a user. The external reader may be used by an authorized third-party-user to scan a cheek portion or mouth of a user and retrieve personal information on the user. The device may be used for a variety of purposes including locating lost or missing people, identifying dead people, retrieving bank information, health history or other important information in emergencies.

The dental lab procedure may include a technician implanting the microchip into a denture or other dental prosthetic item using proper materials. The technician grinds a cavity into a base of the denture or other dental prosthetic item and inserts the microchip within the cavity. Adhesives and acrylic materials may be used to secure and conceal the microchip properly. The dental prosthetic item may then be inserted into the mouth of a user. The information stored on the microchip may then be accessed later by a third party-user using the external reader scanner 130. The microchip, as manufacture and installed, is protected from damage caused by moisture.

A method of using a microchip denture implant and reader system is also disclosed herein. The method of using the microchip denture implant and reader system may comprise the steps of providing a microchip and an external reader scanner 130; entering and programming the microchip with information relating to a user; preparing a cavity to install the microchip within a dental prosthetic item; installing the microchip within the dental prosthetic item; installing the dental prosthetic item in a mouth of the user; scanning the microchip; and accessing the information relating to the user. The method may further comprise the step of updating the information on the microchip, removing the dental prosthetic item, identifying the user, authorizing a third party-user. The dental prosthetic item comprises a denture, in some embodiments.

For purposes of summarizing the invention, certain aspects, advantages, and novel features of the invention have been described herein. It is to be understood that not necessarily all such advantages may be achieved by any one embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description. In light of the devices disclosed in the known art, it is submitted that the present invention substantially diverges in design elements and methods from the known art and consequently it is clear that there is a need in the art for an improvement for microchips and dental prosthetics. In this regard the instant invention substantially fulfills these needs.

### BRIEF DESCRIPTION OF THE DRAWINGS

The figures which accompany the written portion of this specification illustrate embodiments and methods of use for the present disclosure, a microchip denture implant and reader system and method, constructed and operative according to the teachings of the present disclosure.

FIG. 1 is a perspective view of the microchip denture implant and reader system during an ‘in-use’ condition, according to an embodiment of the disclosure.

FIG. 2 is a perspective view of the microchip denture implant and reader system of FIG. 1, according to an embodiment of the present disclosure.

FIG. 3 is a perspective view of the microchip denture implant and reader system of FIG. 1, according to an embodiment of the present disclosure.

FIG. 4 is a perspective view of the microchip denture implant and reader system of FIG. 1, according to an embodiment of the present disclosure.

FIG. 5 is a flow diagram illustrating a method of use for the microchip denture implant and reader system, according to an embodiment of the present disclosure.

FIG. 6 is a schematic diagram of a microchip denture implant.

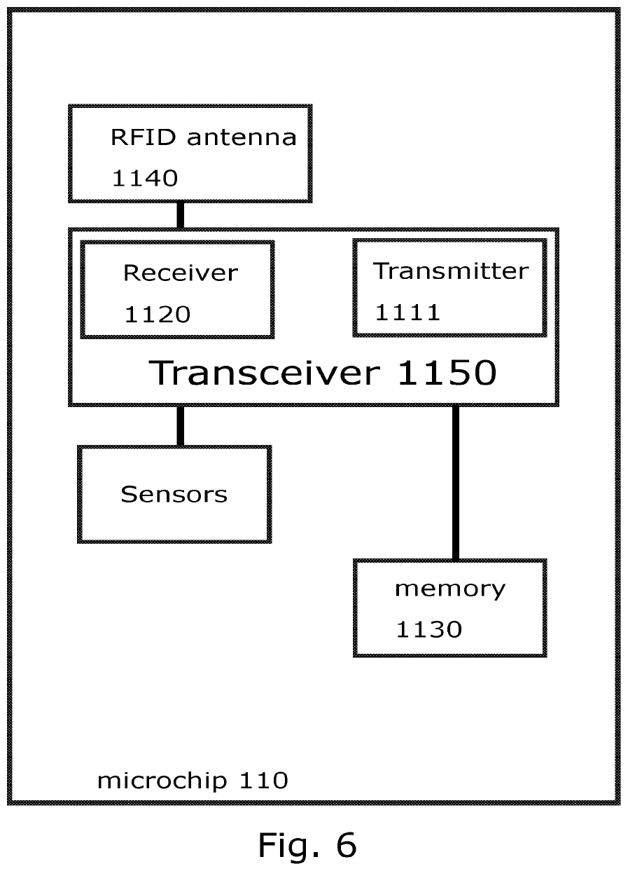

The various embodiments of the present invention will from now on be described with the appended drawings, wherein like designations denote like elements.

### DETAILED DESCRIPTION

As discussed above, embodiments of the present disclosure relate to dentistry and more particularly to a microchip denture implant and reader system and method as used to improve the use of dental accessories and tracking devices in combination.

Generally speaking, the microchip denture implant and reader system include a customized electronic microchip that may be as small as 5 mm. The microchip may be personalized and inserted into a person's denture (prosthetics or any device a person may wear inside the mouth) and include all desired personal information to be accessed when necessary by an external reader device. Personalized, microchip denture implant will be processed inside part of any type of prosthetics wearable inside person's mouth and will be accessed by an external reader device by tapping or scanning through face (cheek area) or mouth of a person or prosthetic device (denture) itself to access information required.

The device may be used for emergency access of a person's personal information, and preferably the access and installation are agreed to by the host. A quick identification may be made on a lost or abducted person or a dead body. Personal information may include passport, identity, driver's license, social insurance, social security, and accounts. The device can operate in various applications including with patients in emergency rooms, hospitals, or other medical facility; as voter registration; by police and for security clearance; for health history information retrieval (medication, allergy, infective disease, and contact information); to locate lost and abducted seniors, seniors with dementia or Alzheimer's; to help deaf and speechless people; to help people with down syndrome or people needing social assistance; to verify club memberships, bank information, and library access; to pay for grocery shopping; to monitor body temperature and other physical information; and many other applications as suitable. The microchip is customizable and personalized for user needs. Information may be programmed by connecting the microchip to a computer via a chip reader cable, such as a USE-chip reader.

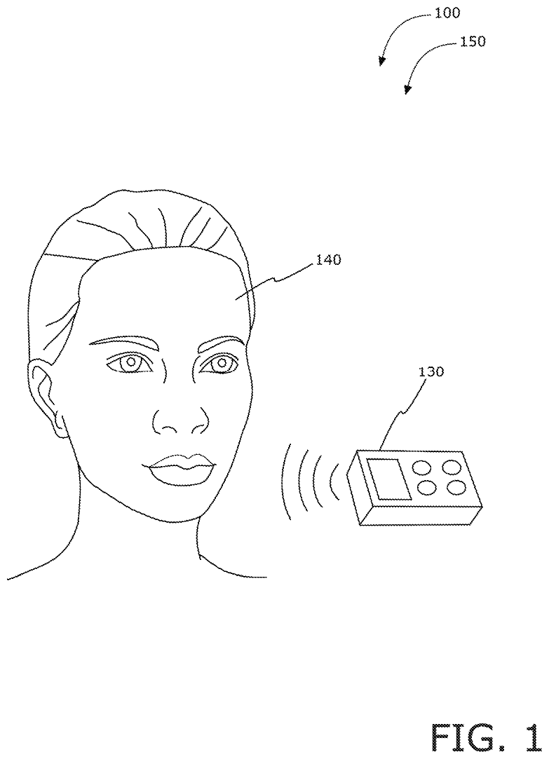

FIGS. 1-4 show various views of a microchip denture implant and reader system 100. FIG. 1 shows a microchip denture implant and reader system 100 during an ‘in-use’ condition 150, according to an embodiment of the present disclosure. As illustrated, the microchip denture implant and reader system 100 may include a microchip 110 having a transceiver 1150 for transmitting a wireless signal containing information relating to a user 140, and an external reader scanner 130 in communication with the microchip 110. In some embodiments, the transceiver 1150 can be a separate receiver and transmitter. The microchip 110 mounts in a dental prosthetic item 10 to be applied to a mouth of or worn by the user 140. The external reader 130 when activated is configured to scan and display or otherwise provide access to the information relating to the user 140. The microchip 110 and dental prosthetic item 10 are configured to provide access to information relating to the user 140 at a given time. Sometimes, this information is available to the microchip when the dental prosthetic item 10 is being worn or used by the user 140. In some embodiments, the device is in communication with a global positioning system to obtain geographic location information relating to the physical location of a user 140. In some of these embodiments, the release or communication of geographic location information is strictly under the user's control. In some embodiments, the external reader/external readers scanner 130 includes a mobile device, such as a smartphone. In other embodiments, the external reader/external readers scanner 130 includes any device configured for wireless communication with the microchip.

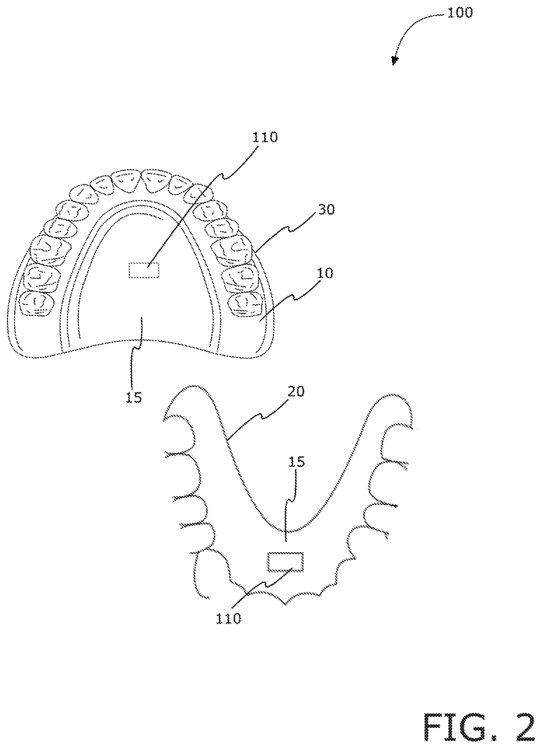

Referring now to FIG. 2 showing a perspective view of the microchip denture implant and reader system 100 of FIG. 1, according to an embodiment of the present disclosure. As above, the microchip denture implant and reader system 100 may include the microchip 110 mounted in the user's 140 dental prosthetic item 10. In various embodiments, the dental prosthetic item 10 includes a partial-denture 20, a complete-denture 30, or other suitable orthodontic or orthopedic appliance. The dental prosthetic item 10 is available as a permanent or removable device, dental implant supported or not. The external reader scanner 130 comprises a display and a control panel for third party-user manipulation. The external reader scanner 130 is portable. The external reader scanner 130 is configured to identify a plurality of different users 140.

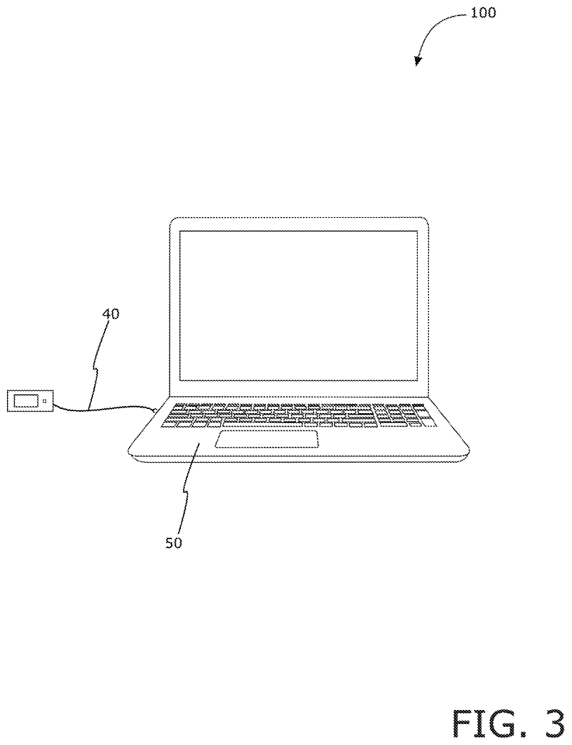

FIG. 3 shows a perspective view of the microchip denture implant and reader system 100 of FIG. 1, according to an embodiment of the present disclosure. As above, the microchip denture implant and reader system 100 may include the microchip 110 including the transceiver 1150 for transmitting a wireless signal containing information relating to the user 140, and the external reader scanner 130 is in communication with the microchip 130. The microchip 130 and the transceiver 1150 are configured to operate within the global positioning satellite system for determining the geographical location of the user 140 under the user's control, in some embodiments. In some embodiments, the device provides an intra-oral tracking means useful for locating missing or lost users 140. The microchip 110 is configured to be personalized and store information relating to the user 140.

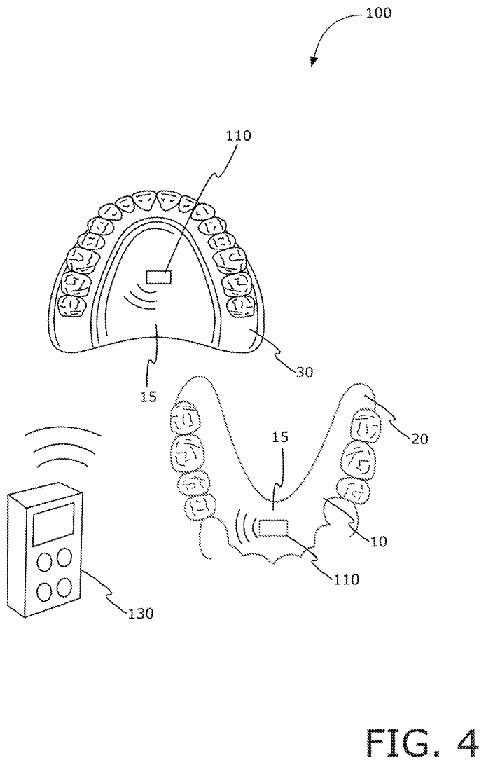

Referring now to FIG. 4 showing a perspective view of the microchip denture implant and reader system 100 of FIG. 1, according. to an embodiment of the present disclosure. In some embodiments, the dental prosthetic item 10 comprises a base 15. As above, the microchip denture implant and reader system 100 has embodiments that include the microchip 110 mounted on base 15. The microchip 110 has a length and a width of approximately 4 to 6 millimeters. The microchip 110 has functionality for storing personalized information or personalize functionality and for receiving information using a USB chip reader m communication with a computer 50 through a cable 40. The user or third-party user may load the microchip 110 with data and update the data accordingly, as needed. In some embodiments, the microchip 110 further includes one or more sensors adapted to measure and record various physiological conditions, including but not limited to body temperature, heart rate, blood pressure, oxygen saturation in the blood, and blood sugar/glucose concentrations. In some embodiments, the captured physiological data is stored on the non-transitory memory 1130 of the microchip 110. The physiological data also includes other metainformation such as the time, sensor make/model, and the like. In some embodiments, the microchip 110 is linked with a computer implemented program, such as a software application of the external scanner 130, or a website accessible from a remote computing device (including the external scanner 130). In one embodiment, the software application allows for access to the non-transitory memory 1130 of the microchip 110. In this way, the scanning of the microchip 110 automatically accesses information stored thereon and provides for viewing of the information stored thereon. In one exemplary use, the physiological data is stored on the non-transitory memory 1130 is transmitted to the software application of the external scanner 130, wherein the data is viewable on a display and formattable by the user.

In some method-based embodiments, a technician takes a dental prosthetic item 10 (necessarily customized to fit the wearer) and modifies the dental prosthetic item 10 to contain the microchip 110. In some embodiments, the modification step has several steps such as milling a space for the microchip 110 in the dental prosthetic item 10. After that, the technician places or inserts the microchip 110 into the milled space and then fixes the microchip 110 in place. In some embodiments, fixing the microchip 110 in place includes gluing or sealing the microchip 110 into the milled space. In some embodiments, the “glue” includes a 2-part resin or a light-activated (e.g., UV) resin or adhesive. Microchip 110 programming can occur before or after placement in the milled space. At any time after programming, the reader 130 can be used to interrogate the microchip 110 either as the dental prosthetic item 10 is worn or after the dental prosthetic item 10 is removed from the user 140.

Some invention embodiments are kits containing the components used to carry out the methods described above. For instance, in some embodiments, the kit comprises a microchip 110 with a microchip transmitter 1111, a microchip receiver 1120, and a microchip memory 1130 and a dental-prosthetic-grade adhesive. Depending upon the requirements the transmitter and receiver can be combined into a transceiver 1150. In some embodiments the microchip 110 is configured for affixation to a dental-prosthetic 10, the receiver is configured to receive instructions and information from an external reader scanner 130, and the transmitter is configured to send information to an external reader scanner 130.

Other embodiments include kits that, in addition to that described above, include an external reader scanner 130 with a scanner transmitter, a scanner receiver, a scanner memory. This scanner 130 can be an RFID scanner that can send commands and information to the microchip 110 and that can receive information from the microchip 110.

In some embodiments, the microchip 110, which can also have RFID capability including an RFID antenna 1140, responds to interrogation from the scanner 130 to provide or transmit information to the scanner 130. These devices can be configured so that they cannot be interrogated from long distances. Some embodiments do not have the ability to provide location data. This can be for privacy reasons.

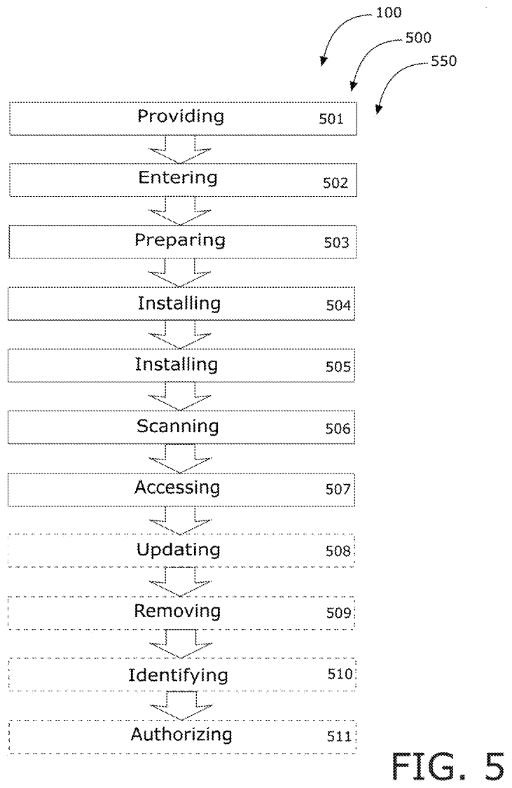

FIG. 5 is a flow diagram 550 illustrating a method 500 of using the microchip denture implant and reader system 100, according to an embodiment of the present disclosure. As illustrated, a method 500 of using the microchip denture implant and reader system 100 may include step one 501, providing a microchip 110 and an external reader scanner 130; step two 502, entering and programming the microchip 110 with information relating to a user 140; step three 503, preparing a cavity to install the microchip 110 within a dental prosthetic item 10; step four 504, installing the microchip 110 within the dental prosthetic item 10; step five 505, installing the dental prosthetic item 10 in the user's 140 mouth; step six 506, scanning the microchip 110; step seven 507, accessing the information relating to the user 140. The method further comprises the step of step eight 508, updating the information on the microchip 110, step nine 509, removing the dental prosthetic item 10, step ten 510, identifying the user 140, and step eleven 511, authorizing a third-party practitioner (home care, hospital, medical or dental clinic, ambulance, nurse, family, etc.). The dental prosthetic item 10 can be a denture.

Steps eight 508, nine 509, ten 510, and eleven 511 are optional, and all embodiments may not implement them. FIG. 5 uses dotted lines to illustrate optional steps of method 500 are illustrated using dotted lines in FIG. 5 to distinguish them from the other steps of method 500. The steps described in the method can be carried out in many different orders according to user preference. The use of “step of” should not be interpreted as “step for”, in the claims herein and is not intended to invoke the provisions of 35 U.S.C. 112(f). Under appropriate circumstances, considering such issues as design preference, user preferences, marketing preferences, cost, structural requirements, available materials, technological advances, etc., other methods for using the microchip denture implant and reader system 100 (e.g., different step orders within above-mentioned list, elimination, or addition of some steps, including or excluding maintenance steps, etc.), are taught herein.

In one embodiment, the denture microchip system a dental prosthetic comprising a milling region adapted to form a milled cavity when a portion of the milling region is removed therefrom. The milted cavity includes an interior volume that is sized to receive a microchip. The microchip is positioned within the milled cavity of the dental prosthetic. The microchip includes a transmitter configured to send information from a memory to an external reader scanner. In this way, the dental prosthetic information may be stored on the microchip. The transmitter is adapted to transmit, receive, or transmit and receive information via signal.

A dental prosthetic grade adhesive adapted to close an open end of the milled cavity such that the microchip is secured within the milled cavity. The dental prosthetic grade adhesive thereby conceals the microchip from exterior view, improving aesthetic appeal of the prosthetic. In one embodiment, the dental prosthetic grade adhesive is disposed within the milled cavity to conceal the microchip. The dental prosthetic grade adhesive may be transparent, opaque, or the like.

In one embodiment, the milling region is disposed on a non-tooth portion of the dental prosthetic. In another embodiment, the milling region is disposed on a tooth portion of the dental prosthetic.

In one embodiment, the denture microchip kit comprises a microchip including a microchip transmitter and a microchip memory, wherein the microchip is configured for implantation in a milled cavity in a dental prosthetic. The microchip transmitter is configured to send information to an external reader scanner. In this way, the microchip may have information related to the prosthetic stored thereon, such as make, model, year, modification, patient information, and the like. The dental prosthetic includes a milling region having a thickness such that the milled cavity is formed when material is removed therefrom, wherein the milled cavity forms an interior volume sized to receive the microchip. A dental prosthetic grade adhesive adapted to secure the microchip within the milled cavity, wherein the dental prosthetic grade adhesive is adapted to seal the milled cavity. In this kit, the microchip and dental prosthetic are packaged together for implementation by the dental professional upon receipt. This provides for selective customization and sizing for each patient.

In one embodiment, the present invention includes a method of implanting a microchip to a denture prosthetic, comprising providing the microchip having a transmitter configured to send information from a memory to an external reader scanner, providing a dental prosthetic, the dental prosthetic comprising a milling region, and milling the milling region to form a milled cavity, wherein the milled cavity is dimensioned to receive the microchip. Moreover, the method may include any of the following: applying a dental prosthetic grade adhesive to close an open end of the milled cavity such that the microchip is secured within the milled cavity, installing the microchip within the milled cavity of the dental prosthetic, implanting the dental prosthetic in a mouth of a user before the microchip is installed within the milled cavity, implanting the dental prosthetic in a mouth of a user after the microchip is installed within the milled cavity, and scanning the microchip with the external reader scanner to receive information therefrom.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A denture microchip kit, comprising:
a microchip including a microchip transmitter and a microchip memory;
wherein the microchip is configured for implantation in a milled cavity in a dental prosthetic;
wherein the microchip transmitter is configured to send information to an external reader scanner;
the dental prosthetic comprising a milling region having a thickness such that the milled cavity is formed when material is removed therefrom, wherein the milled cavity forms an interior volume sized to receive the microchip;
a dental adhesive adapted to secure the microchip within the milled cavity, wherein the dental adhesive is adapted to seal the milled cavity.

2. The kit of claim 1, wherein the external reader scanner includes a scanner transceiver and a scanner memory, wherein the scanner transceiver is configured to receive information from the microchip transmitter.

3. The kit of claim 2, wherein the microchip is configured to transmit information from microchip memory.

4. The kit of claim 1, wherein the microchip has a length and a width of approximately 4 to 6 millimeters.

5. The kit of claim 1, wherein the milling region is disposed on a tooth portion of the dental prosthetic.

6. The kit of claim 1, wherein the milling region is disposed on a non-tooth portion of the dental prosthetic.

7. The kit of claim 1, wherein the milling region is disposed on an exterior side of the dental prosthetic.

8. The kit of claim 1, wherein the milling region is disposed on an interior side of the dental prosthetic.

9. A denture microchip system, comprising:
a dental prosthetic comprising a milling region adapted to form a milled cavity when a portion of the milling region is removed therefrom;
wherein the milted cavity includes an interior volume that is sized to receive a microchip;
the microchip disposed within the milled cavity of the dental prosthetic, the microchip including:
a transmitter configured to send information from a memory to an external reader scanner;
a dental adhesive adapted to close an open end of the milled cavity such that the microchip is secured within the milled cavity.

10. The kit of claim 9, wherein the milling region is disposed on a tooth portion of the dental prosthetic.

11. The kit of claim 9, wherein the milling region is disposed on a non-tooth portion of the dental prosthetic.

12. The kit of claim 9, wherein the milling region is disposed on an exterior side of the dental prosthetic.

13. The kit of claim 9, wherein the milling region is disposed on an interior side of the dental prosthetic.

14. A method of implanting a microchip to a denture prosthetic, comprising:
providing the microchip comprising a transmitter configured to send information from a memory to an external reader scanner;
providing a dental prosthetic, the dental prosthetic comprising a milling region;
milling the milling region to form a milled cavity, wherein the milled cavity is dimensioned to receive the microchip.

15. The method of claim 14, further comprising:
applying a dental adhesive to close an open end of the milled cavity such that the microchip is secured within the milled cavity.

16. The method of claim 15, further comprising:
installing the microchip within the milled cavity of the dental prosthetic.

17. The method of claim 16, further comprising:
implanting the dental prosthetic in a mouth of a user before the microchip is installed within the milled cavity.

18. The method of claim 16, further comprising:
implanting the dental prosthetic in a mouth of a user after the microchip is installed within the milled cavity.

19. The method of claim 16, further comprising:
scanning the microchip with the external reader scanner to receive information therefrom.
